(19) United States
(12) Patent Application Publication
(10) Pub. No.: US 2024/0238378 A1
(43) Pub. Date: Jul. 18, 2024

(54) METHODS AND CCK-B RECEPTOR AGONISTS FOR TREATING TINNITUS

(71) Applicant: City University of Hong Kong, Kowloon (HK)

(72) Inventors: Jufang He, Kowloon Tong (HK); Peipei Zhou, Shatin (HK); Zhoujian Xiao, Kwun Tong (HK); Xi Chen, Kwun Tong (HK)

(21) Appl. No.: 18/405,103

(22) Filed: Jan. 5, 2024

Related U.S. Application Data
(60) Provisional application No. 63/437,436, filed on Jan. 6, 2023.

(51) Int. Cl. A61K 38/22; A61P 27/16

(52) U.S. Cl. CPC A61K 38/2207; A61P 27/16

(57) ABSTRACT

A method of alleviation or therapy of tinnitus in a subject in need thereof includes the step of administrating a CCK-B receptor agonist to the subject. A CCK-B receptor agonist is used for alleviation or therapy of tinnitus in a subject in need thereof.

The current application claims a priority to the U.S. Provisional Patent application Ser. No. 63/437,436 filed on Jan. 6, 2023.

### FIELD OF THE INVENTION

The present invention relates to a method and a CCK-B receptor agonist for treating tinnitus.

### BACKGROUND

Tinnitus is one of the common medical symptoms of many diseases. Despite the prevalence estimations scatter widely, the prevalence of tinnitus in adult is already more than 10% not only in developed countries such as the USA and Europe but also in middle-income and low-income developing countries in Asia and Africa. This indicates tinnitus is a global burden. As per the US Centers for Disease Control (CDC), around 15% of the population of the U.S., or 50 million people, suffer from some form of tinnitus. Chronic tinnitus is estimated to affect around 20 million people in the US. According to estimates by the Chinese Federation of People with Disabilities, approximately 130 million Chinese experience tinnitus sounds in their ears. These are the patients who are disturbed enough to seek medical evaluation, and therefore the number is probably much lower than the actual number of tinnitus sufferers. Because of demographic developments and an increase in professional and leisure noise exposure, tinnitus prevalence is expected to continue to increase. Moreover, tinnitus is among the most frequent sequelae of modern warfare.

The world's elderly population is rapidly growing, which creates a huge potential for the growth of the global tinnitus market. Aging is a major contributor to hearing loss which is the predominant cause of tinnitus. According to the report of World Health Organization (WHO), the number of elderly (aged 60 years and above) was 1 billion in 2019. The elderly population is expected to double in number and will increase to 2.1 billion in 2050. Hence, the increasing prevalence of tinnitus around the globe is likely to boost the growth of the tinnitus management market in the future.

The primary treatment for tinnitus is sound therapy and cognitive-behavioral therapy, but while these treatments might relieve tinnitus, they do not cure it. A therapeutic brain-machine interface could be more effective while it is less-flexible, and less-biocompatible.

Chinese application CN 113929737 A by Tortorella, et al. to Regenerative Medicine and Health Innovation Centre Co., Ltd., et al., published on Jan. 14, 2022, discloses a polypeptide and application thereof as a CCK receptor agonist/antagonist. However, it does not mention the use of the CCK receptor agonist for treating tinnitus.

The American government and some Tinnitus Associations like the American Tinnitus Association (ATA) and the British Tinnitus Association (BTA) have granted a huge amount of money in funding for tinnitus research since 1980. Unfortunately, there are no effective medications that treat tinnitus. Despite the mechanism and possible treatments for tinnitus having made great progress, hitherto, there is no effective drug approved by The United States Food and Drug Administration (FDA) for treatment.

Therefore, there remains a need for new therapies for potential prophylaxis, treatment and curing of tinnitus.

### SUMMARY OF THE INVENTION

An embodiment of this invention relates to a method of alleviation or therapy of tinnitus in a subject in need thereof, including the step of administrating a CCK-B receptor agonist to the subject.

An embodiment of the present invention relates to relates to use of a CCK-B receptor agonist for alleviation or therapy of tinnitus in a subject in need thereof.

An embodiment of the present invention relates to the use of a CCK-B receptor agonist in the manufacture of a medicament for the alleviation or therapy of tinnitus in a subject in need thereof.

Without intending to be limited by theory, it is believed that the present invention may provide a novel approach for alleviation or therapy of tinnitus that targets CCK-B receptor. Furthermore, it is believed that the present invention may possess fewer side effects than other potential treatments, which, for example, target NMDA receptors.

### BRIEF DESCRIPTION OF THE DRAWINGS

FIG. 1 shows calcium response (ΔF/F0) measured at various HT-267 concentrations in GPCRs overexpressed CHO cells, including CHO-CCKBR, CHO-CCKAR, CHO-HTR2B cells;

FIG. 2A is a diagram showing the schematic of gap pre-pulse inhibition of the acoustic startle reflex (GPIAS) setup;

FIG. 2B is a diagram showing the principle of GPIAS;

FIG. 2C is a bar chart showing changes of GPIAS ratios before the day of noise exposure (pre-NE) and 7 weeks after the day of noise exposure (7 weeks post-NE) at a frequency of 9k Hz:

FIG. 2D is a bar chart showing changes of GPIAS ratios before the day of noise exposure (pre-NE) and 7 weeks after the day of noise exposure (7 weeks post-NE) at a frequency of 12.6k Hz:

FIG. 2E is a bar chart showing changes of GPIAS ratios before the day of noise exposure (pre-NE) and 7 weeks after the day of noise exposure (7 weeks post-NE) at a frequency of 16k Hz:

FIG. 2F is a bar chart showing changes of GPIAS ratios before the day of noise exposure (pre-NE) and 7 weeks after the day of noise exposure (7 weeks post-NE) at a frequency of 20.1k Hz:

FIG. 2G is a bar chart showing changes of GPIAS ratios before the day of noise exposure (pre-NE) and 7 weeks after the day of noise exposure (7 weeks post-NE) at a frequency of 28.7k Hz:

FIG. 3A is a schematic diagram representing the experimental setup (left) and the Nissl stained brain slice (right) showing the position of the ES electrode tips in the MGv, as indicated by the asterisk, Scale bar: 500 μm;

FIG. 3B is a diagram showing raw signal of the extracellular multiunit recording in MGv and ACx;

FIG. 3C is a diagram showing input-output relationship between stimulation intensity in MGv and evoked fEPSP in ACx;

FIG. 3D is a diagram showing HFS-induced LTP in the auditory thalamocortical pathway: population data of normalized fEPSP slopes before and after HFS (n=11, One-way ANOVA, p<0.001), and sample fEPSP waveforms before and after HFS (1, 2); scale bar: 5 ms, 0.2 mV;

FIG. 3E is a diagram showing that CCK injection in the auditory cortex induces thalamocortical LTP;

FIG. 4A is a diagram showing the preparation (upper) and the paradigm (lower) of the test for a natural auditory response;

FIG. 4B are charts showing neuronal responses in raster displays and peristimulus time histograms in the ACx before and after HFS in the MGB;

FIG. 4C is a graph showing Z-score (mean±SEM) of neuronal responses to the noise bursts in the ACx before (grey) and after HFS (dark grey) in the MGB (Bin width, 5 ms, n=12, two-way ANOVA, p<0.001; post-hoc, Tukey test, before vs. after HFS, **, p<0.001, *, p<0.05);

FIG. 5A are images showing immunostained CCKBR expression of primary Auditory Cortex (ACx) (upper); and CCK BR expression of Layer3/4 of ACx where clustered the terminals of thalamocortical projection neurons (lower), Blue—DAPI, Magenta—Anti-CCKBR. Upper Scale bar: 200 μm;

FIG. 5B is a diagram showing the experiment design (upper); and the experimental protocol (lower);

FIG. 5C is a graph showing normalized fEPSP slope % with respect to time for the control group and the HT-267 injected group;

FIG. 6A is a diagram showing schematic of HT-267 mediated sound therapy;

FIG. 6B is a bar chart showing changes of GPIAS ratios pre- and post-treated with control solution combined with sound therapy at a frequency of 12.6k Hz;

FIG. 6C is a bar chart showing changes of GPIAS ratios pre- and post-treated with control solution combined with sound therapy at a frequency of 16k Hz;

FIG. 6D is a bar chart showing changes of GPIAS ratio pre- and post-treated with control solution combined with sound therapy at a frequency of 20.1k Hz;

FIG. 6E is a bar chart showing changes of GPIAS ratio pre- and post-treated with control solution combined with sound therapy at a frequency of 28.7k Hz;

FIG. 6F is a bar chart showing changes of GPIAS ratio pre- and post-treated with HT-267 combined with sound therapy at a frequency of 12.6k Hz;

FIG. 6G is a bar chart showing changes of GPIAS ratio pre- and post-treated with HT-267 combined with sound therapy at a frequency of 16k Hz;

FIG. 6H is a bar chart showing changes of GPIAS ratio pre- and post-treated with HT-267 combined with sound therapy at a frequency of 20.1k Hz; and

FIG. 6I is a bar chart showing changes of GPIAS ratio pre- and post-treated with HT-267 combined with sound therapy at a frequency of 28.7k Hz.

The figures herein are for illustrative purposes only and are not necessarily drawn to scale.

### DESCRIPTION OF THE PREFERRED EMBODIMENTS

Unless otherwise specifically provided, all tests herein are conducted at standard conditions which include a room and testing temperature of 25° C., sea level (1 atm.) pressure, pH 7, and all measurements are made in metric units. It is understood that unless otherwise specifically noted, the materials compounds, chemicals, etc. described herein are typically commodity items and/or industry-standard items available from a variety of suppliers worldwide.

Tinnitus, a common complication in other diseases and otological symptoms in patients, affects at least 10% of the population around the world. Patients are aware of sharp and annoying ringing sounds even when an external auditory stimulus is absent. Most people have experienced tinnitus. The cause of tinnitus is diverse and complicated. For instance, it is believed that impairment of the auditory pathway, COVID-19, and some mental diseases may induce tinnitus. Usually, elderly people have a higher risk than young people due to longer-time noise exposure, but tinnitus occurs in young people more often these days. Although some therapeutic approaches that aim to change neural plasticity can relieve tinnitus, there is no available drug that can treat tinnitus. Many people experience great suffering from tinnitus.

Current research and development in the treatment of tinnitus focuses on neuroplastic changes. The main non-drug treatments used in clinic are rTMS and DBS. For medication, there are no FDA-approved drugs specifically for tinnitus. Some tinnitus studies suggest that drugs that target neurotransmitter receptors, such as NMDA receptor antagonists, can reduce tinnitus. However, NMDA receptors are widely expressed in the brain, resulting in antagonists that can have significant side effects. And the agonist proposed in this invention is targeting a neuromodulatory receptor—Cholecystokinin B receptor (CCKBR), which the inventors believe is a new target for the treatment of tinnitus. CCKBR is not as widely distributed in the brain as NMDA receptors, therefore it is believed that a therapy targeting CCKBR-targeting drug may cause fewer side effects.

The treatment strategy employed in this application is built upon the inventor's extensive research conducted over the past decade, which aimed to elucidate the fundamental properties and functions of cholecystokinin (CCK) within the central nervous system. A pivotal contribution to this project came from our previous studies (Li X, et al., Cholecystokinin from the entorhinal cortex enables neural plasticity in the auditory cortex. Cell Res. 2014; 24(3):307-30), wherein they proposed that CCK-positive projections originating from the Entorhinal cortex (EC) play a crucial role in facilitating neural plasticity in the auditory cortex (AC). CCK8 s were injected at the recording site of AC locally through an injection cannula and the characteristic frequencies at this site were acoustically paired with weak response frequencies, and following this pairing, responses to weak response frequencies were enhanced. Chen et al (Chen X, et al. Cholecystokinin release triggered by NMDA receptors produces LTP and sound-sound associative memory. Proc Natl Acad Sci USA. 2019; 116(13):6397-406.) demonstrated that the release of CCK is mediated by NMDA receptors, and the activation of CCKB receptors (CCKBR) is essential for the induction of high-frequency stimulation (HFS)-induced long-term potentiation (LTP). Through a loss-of-function experiment, they successfully blocked CCKBR using a specific antagonist, resulting in the inability to achieve HFS-induced LTP in the auditory cortex of mice lacking CCK. The present application further reinforces that CCK plays a critical role in triggering plasticity that is contingent upon the activation of CCKBR in the auditory cortex. Without intending to be bound by theory, it is believed that this process according to this invention strengthens synaptic projections from tonotopically neighboring areas towards the hyperactive region induced by tinnitus, leading to a restructuring of the auditory system and ultimately alleviating tinnitus symptoms.

An embodiment of the present invention relates to a method of alleviation or therapy of tinnitus in a subject in need thereof, including the step of administrating a Cholecystokinin B (CCK-B) receptor agonist to the subject.

The present invention surprisingly finds that CCK-B receptor agonist (such as HT-267, a cholecystokinin-4 modified compound) combined with sound exposure for noise-induced tinnitus animals successfully reduces gap pre-pulse inhibition of the acoustic startle reflex (GPIAS) that evaluate the presence of tinnitus. This is the first time that HT-267—a CCK B receptor agonist is proposed and validated for treating tinnitus in a subject. Without intending to be limited by theory, it is believed that medicaments for treating tinnitus are required to penetrate the blood-brain barrier (BBB) to enter the brain where the medicament exerts its function. The CCK-B receptor agonist used herein (such as HT-267) passes BBB easily and can be administrated orally and has a high affinity to the CCK-B receptor in the brain, which could lead to neuroplastic changes and relieves tinnitus. Compared with other treatments, it is believed that oral administration of HT-267 may be biocompatible, more flexible, not invasive, and have a favorable therapeutic effect.

In an embodiment herein, the CCK-B receptor agonist is selected from HT-267 having the following formula (I):

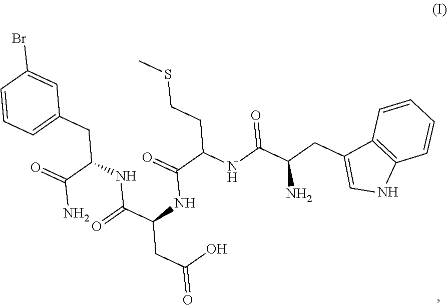 [SMILES: CSCCC(NC(=O)[C@H](N)Cc1c[nH]c2ccccc12)C(=O)N[C@@H](CC(=O)O)C(=O)N[C@@H](Cc1cccc(Br)c1)C(N)=O]

CCK-4 having the following formula (II):

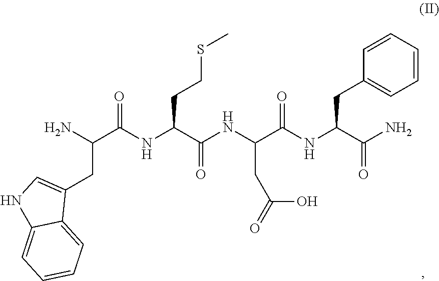 [SMILES: CSCC[C@H](NC(=O)C(N)Cc1c[nH]c2ccccc12)C(=O)NC(CC(=O)O)C(=O)N[C@@H](Cc1ccccc1)C(N)=O]

or a mixture thereof.

Cholecystokinin-4 (CCK-4) is known as a CCK-B receptor (CCKBR) agonist, a truncated version of CCK, and it consists of 4 amino acids (Trp-Met-Asp-Phe) and an amino group (—NH₂), as shown in formula (II).

The structure of HT-267 is similar to the structure of CCK-4. Without intending to be limited by theory, it is believed that the modification on HT-267 improves the stability and activity thereof by introducing halogens —Br at the third position of the phenyl group in phenylalanine. CN113929737A (Tortorella et al.) discloses the structure of HT-267 but it does not mention the use of HT-267 for the therapy of tinnitus. In addition, it is surprisingly found that HT-267 may relieve tinnitus, which suggests that CCKBR can be a new drug target for the treatment of tinnitus.

In some embodiments, the Tmax (Time to peak drug concentration) of HT-267 is about 0.033 h, which demonstrates it takes less time for HT-267 to reach the Cmax (maximum concentration) after administration of the compound and it can be absorbed quickly.

In some embodiments, the elimination half-life (t_{1/2}) of HT-267 (about 1.745 h) in plasma is much longer than t_{1/2} of CCK-4 (about 13 minutes), and in consequence, HT-267 stays in the body for a longer time, prolonging its effect.

In some embodiments, pharmacokinetic parameters for HT-267 in brain show that the uptake rate of brain is about 21.89%, which indicates that HT-267 may have a high absorption rate and bioavailability. Also, the t_{1/2} of HT-267 in brain is up to about 8.701 hours, which means the drug may maintain the effective concentration in the brain for a long period of time.

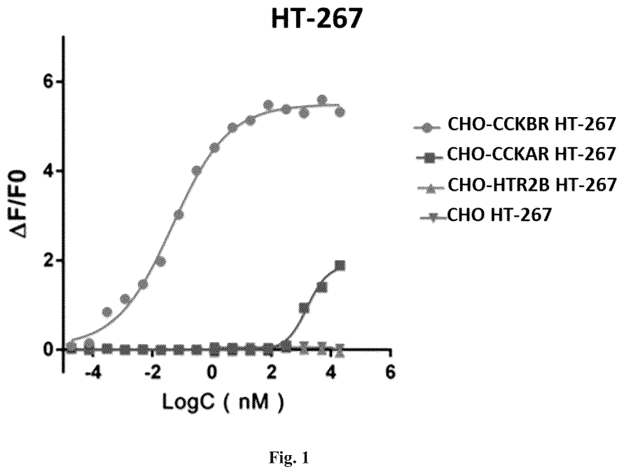

In vitro screening system, HT-267 have been tested and measured calcium response by Fluo-8 No Wash Calcium Assay Kit in GPCRs overexpressed CHO cells, including CHO-CCKBR, CHO-CCKAR, CHO-HTR2B cells. These tests and measurements are performed as described in CN 113929737 A by Tortorella, et al., assigned to Regenerative Medicine and Health Innovation Centre Co., Ltd., et al., published on Jan. 14, 2022. Data shows that HT-267 specifically activates CCKBR (FIG. 1), the EC50 of HT-267 is about 0.05 nM, which implies that HT-267 may have a higher potency than CCK-4.

In an embodiment herein, the method further includes subjecting the subject to a sound exposure after administrating the CCK-B receptor agonist. For example, the sound lasts about 1400 ms, which consists of 100 ms white noise (start at 150 ms and 600 ms) and 100 ms 5 merged frequencies (start at 250 ms and 500 ms). In some embodiment, the subject is exposed to the sound for about 12 minutes.

In an embodiment herein, the administrating step includes a route selected from the group of oral administration, intraperitoneal administration, intravenous administration, and a combination thereof.

In some embodiments, the CCK-B receptor agonist is administrated via intravenous administration. Without intending to be limited by theory, it is believed that via intravenous administration the agonist can bypass first-pass metabolism and result in higher bioavailability than intraperitoneal administration.

In some embodiments, the CCK-B receptor agonist is administrated via intraperitoneal administration. Without intending to be limited by theory, it is believed that intraperitoneal administration can avoid the gastrointestinal tract and potential degradation of the agonist that may happen for oral administration.

In an embodiment herein, the CCK-B receptor agonist is administrated during a period of about a week, for example, every two days.

In an embodiment herein, the CCK-B receptor agonist is administrated at a dosage of from about 0.1 mg/kg to about 5 mg/kg. For example, the agonist is administrated at a dosage of about 0.15 mg/kg, about 0.2 mg/kg, about 0.5 mg/kg, about 1 mg/kg, about 2 mg/kg, about 3 mg/kg, or about 4 mg/kg. In some embodiment, the CCK-B receptor agonist is administrated at a dosage of about 0.2 mg/kg. Without intending to be limited by theory, it is believed that the CCK-B receptor agonist can function at a low dosage, for example, even of about 0.2 mg/kg.

An embodiment of the present invention relates to use of a CCK-B receptor agonist for alleviation or therapy of tinnitus in a subject in need thereof.

In an embodiment herein, the CCK-B receptor agonist is selected from HT-267 of formula (I), CCK-4 of formula (II), or a mixture thereof.

In an embodiment herein, the CCK-B receptor agonist is in a dosage form suitable for an administration route selected from the group consisting of oral administration, intraperitoneal administration, intravenous administration, and a combination thereof. In some embodiments, the CCK-B receptor agonist is in a dosage form of, for example, powder, solution, suspension or the like.

In an embodiment herein, the CCK-B receptor agonist is dosed in a concentration of about 0.02 wt. %.

An embodiment of the present invention relates to use of a CCK-B receptor agonist in the manufacture of a medicament for alleviation or therapy of tinnitus in a subject in need thereof.

In an embodiment herein, the CCK-B receptor agonist is selected from HT-267 of formula (I), CCK-4 of formula (II), or a mixture thereof.

In an embodiment herein, the medicament is in a dosage form suitable for an administration route selected from the group consisting of oral administration, intraperitoneal administration, intravenous administration, and a combination thereof. In some embodiments, the medicament is in a dosage form of, for example, powder, solution, suspension or the like.

In an embodiment herein, the medicament comprises CCK-B receptor agonist in a concentration of about 0.02 wt. %.

In an embodiment herein, the subject being treated with the method or the medicament herein is a human.

Without intending to be limited by theory, it is believed that excessive exposure to environmental noise is the main cause of cochlear damage, which induces a decrease in the input of auditory nerve fiber from the cochlea to the brain. The cochlear nucleus (CN) is the first station that receives inputs from the cochlea. After noise exposure, the output of cochlea to CN may be decreased while the output of CN, for example, neural spontaneous firing rates and synchrony, may be frequency-specifically increased due to homeostatic or timing-dependent plasticity. It is also believed that the most common consequence of high-frequency hearing loss in animals and tinnitus people is that the cortical neurons in the hearing loss area begin to respond preferentially to the sound frequencies at the edges of normal hearing, resulting in cortical topographic map changes. The reorganization model of the primary auditory cortex map may occur when neurons that receive reduced thalamocortical input begin to respond preferentially to input from their unaffected neighbors via horizontal connections. Because the auditory thalamus and cortex are intensively interconnected, forming a strong thalamocortical feedback loop, this loop may amplify the hyperactivity of the deafferented region through parasitic oscillation theory and ultimately induce tinnitus.

Without intending to be limited by theory, it is believed that cholecystokinin and its B receptors that modulate neuronal activity are extremely abundant in the auditory systems. The present invention demonstrates that activation of the CCK-B receptor can induce Long-term Potentiation (LTP) in the auditory cortex. In comparison with CCK-4 (a well-known CCKBR agonist), HT-267 has higher affinity and better stability. CCKBR agonists such as HT-267 could relieve and treat tinnitus, so that the patient no longer suffers from pain caused by tinnitus, such as insomnia, depression, anxiety, etc. Without intending to be limited by theory, it is believed that one possible theory of why HT-267 treats tinnitus is that CCKBR agonists combined with sound exposure will reshape the plasticity of the thalamus-auditory cortex loop, which increases the input to neurons in the auditory cortex that are overactive due to reduced input. Consequently, the total gain of this loop is below 1, when parasitic oscillation stops which relieves tinnitus. On the other hand, the present invention provides a new target for the development of new drugs for tinnitus. That is, other CCKBR agonists like HT-267 may be effective to treat tinnitus.

Standard treatments for tinnitus in clinic include sound therapy and cognitive-behavioral therapy (CBT). However, both treatments cannot cure tinnitus many times, and they can help make the symptoms of the patient less noticeable. Hearing aids and sound generators are two main methods of sound therapy. Hearing aids that enhance objective and peripheral sounds can provide some relief even if the patient has no obvious hearing loss. The principle of the sound generator is to provide external sound on which patients will focus, which makes tinnitus less obvious. Recently, Aurex International Corp (AIC) developed Aurex3 that is a non-invasive therapy device for tinnitus with a wearable headset that generates a complex sound signal to counteract the patient's tinnitus through a smartphone. Crucially, the introduction of the complex sound signal is used to specifically excite the areas of a patient's tinnitus sound derived from neurophysiological and psychological dysfunction temporarily while the dysfunction of the neural network remains unsolved. Cognitive-behavioral therapy used to treat depression and anxiety can also treat tinnitus since tinnitus is correlated with mental disorders. CBT can help patients to reorganize negative thoughts to bring about positivity and allows the patient to be accustomed to the noise. Besides, some therapeutic brain-machine interfaces can also relieve tinnitus by targeting the neuroplastic changes underlying tinnitus. For instance, Repetitive transcranial magnetic stimulation (rTMS) and Deep brain stimulation (DBS) decrease neuronal activity, which has benefits for tinnitus, but the long-term safety and efficacy of this treatment are still unknown. The disadvantages of these treatments are less biocompatibility, no flexibility, and much invasive.

### EXAMPLES

### 1. High Frequency Stimulation (HFS) of MGB

The electrical stimulation is generated by an ISO-Flex isolator (A.M.P.I., Israel), which is controlled by a multifunction processor (RX6, TDT). The electrical current pulses for baseline test are 0.5 ms, 50-150 μA and presented every 10 s. The theta burst stimulation (TBS) contains four trails of 10 bursts at 5 Hz with the interval of 10 s between the trails, and each burst consists of 5 pulses at 100 Hz.

In TBS-induced thalamocortical LTP experiments, two customized microelectrode arrays with four tungsten electrodes each, impedance: 0.5-1 MS2 (recording), and 200-500 kΩ (ES) (FHC, U.S.), are used for the auditory cortical neuronal activity recording and medial geniculate body ventral division (MGv) electrical stimulation and monitoring, respectively. The two electrode arrays are driven by two micro-manipulators separately. 70 dB, 100 ms, noise bursts are presented every 10 s during the insertion of the electrode arrays, and neuronal activities are monitored as the electrode arrays are lowered until robust noise responses are achieved on both arrays. The final stimulating and recording positions are determined by maximizing the cortical field excitatory postsynaptic potential (fEPSP) amplitude triggered by the electrical stimulation in MGv. Histological confirmation shows that the stimulating electrode tip is placed in MGv.

The fEPSPs, which are elicited by 0.5 ms electrical current pulses, are amplified (×1000) and filtered (1 Hz-5 kHz), recorded with a 25 kHz sampling rate, and stored in a PC by OpenEx software (TDT). Before the recording, an input-output function is measured. A stimulation current, which elicited a fEPSP amplitude 50% of maximum, is chosen for the baseline and after TBS recording. The fEPSPs are collected for 15 min before and 1 hour after TBS, respectively. For TBS, 10 negative electrical current bursts are delivered in 5 Hz and repeated 4 trials. The inter-trial interval is 10 s. Each burst contains 5, 0.5 ms pulses, at 100 Hz, and the current of the pulses is selected 75% of the maximal response from the input-output relationship. The slopes of the evoked fEPSPs are calculated and normalized by customized MATLAB script, and the group data is plotted as mean±SEM.

In the thalamocortical LTP induction experiments, CCK (1 μM, 1 μl) or ACSF, HT-267 (5 μM, 0.5 μl) is injected into the auditory cortex. Low-frequency electrical stimulation, which contains 200, 0.5 ms, 75% saturated current pulses in 0.1 Hz are delivered into MGv after the injection.

### 2. Auditory Stimuli

Auditory stimuli, including pure tones and noise bursts, are generated by Tucker-Davis Technologies (TDT, U.S.) workstation and delivered through an electrostatic speaker (ED1, TDT). The speaker is placed at 10 cm away from the awake animals or directly to the ear contralateral to the implanted electrodes via a hollow ear bar for the anesthetized animals. The sound pressure level of the speaker is calibrated with a microphone (B&K, Denmark).

### 3. Immunohistochemistry-IHC

Animals are deeply anesthetized with high dose of sodium pentobarbital (100 mg/kg, I.P). After that, they are perfused transcardially with 20 ml 0.1 M PBS, followed by 4% (w/v) paraformaldedyde (PFA) in 0.1M PBS. Brains are carefully removed from the skull and post-fixed with 4% (w/v) PFA at 4° C. overnight. Brains are then serially dehydrated in 10% (w/v), 20% (w/v) and 30% (w/v) sucrose. Brains are sectioned to 30 μm coronal slices.

For staining, slices are washed 4 times, 10 min each in PBS. Blocking solution (10% (v/v) Goat serum+0.1% (v/v) Triton-X) is applied and slices are incubated for 2 hours at room temperature. Brain slices are incubated with Primary antibody Anti-CCKBR (ab77077, Abcam) which is diluted in Blocking solution at dilution of 1:1000 at 4° C. overnight. Slices are further rinsed with 1×PBS 5 times at 10 min each time. Slices are incubated with Secondary antibody (Donkey anti-Goat IgG (H+L) Cross-Adsorbed Secondary Antibody, Alexa Fluor 647, A-21447, Invitrogen Antibodies) which is diluted in PBST (0.1% (v/v) Triton-X) at room temperature for 2 hours, followed by incubation with DAPI in PBS for 10 min, and washed 3 times with PBS. Finally, slices are mounted on the glass slide and added glycerol and cover slip. Images are captured through the Nikon AX R Confocal Microscope with a 20× objective lens.

### 4. Drug Preparation

The HT-267 compounds cannot be purchased on the market, and those used herein are synthesized exclusively in the lab of Dr. Micky Tortorella who has the patent for the HT-267 compound.

The purity of HT-267 powder should be >95% (HPLC). HT-267 power is dissolved in Dimethyl sulfoxide (DMSO) with the stock concentration of 10 mM. The drug usually is stored at −20° C. On the day of injection, the stock solution is diluted with saline freshly to the injection concentration—320 μM, including 3.2% (v/v) DMSO and 2% (v/v) Cremophor EL.

### 5. Tinnitus Mouse Model

HT-267 is tested in the tinnitus mouse model. One hour-long pure 12.6 kHz tone will be presented to the unilateral ear of the mouse to induce acoustic trauma and hence the tinnitus in some of the animals. 7-9 weeks after tinnitus induction, more than 50% of mice developed tinnitus.

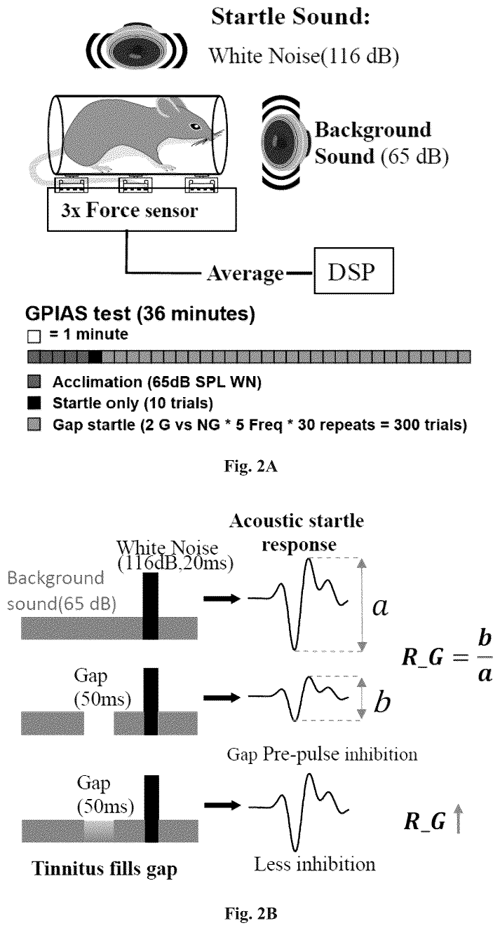

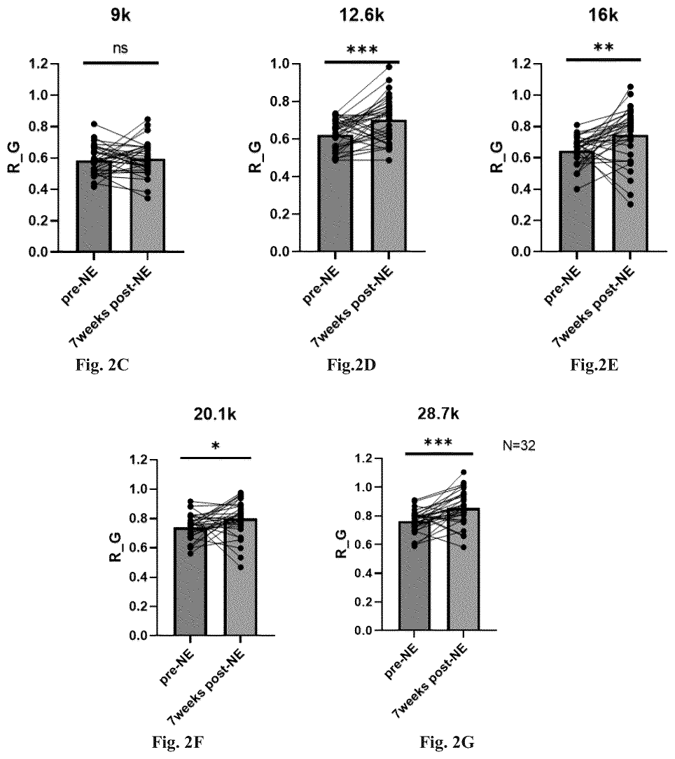

The inventors use the standard approach for assessing tinnitus in a rodent model, namely gap pre-pulse inhibition of the acoustic startle reflex (GPIAS) (FIGS. 2A-B). FIG. 2A shows the schematic of GPIAS setup. (Upper) Background sound and startle speakers are placed 10 and 13 cm respectively, from the point of microphone calibration. Signals from three force sensors placed under the testing tube in a triangular formation are averaged in a custom-made circuit and transmitted to TDT digital signal processor (DSP) for further analysis. (Lower) Timeline of a single GPIAS testing session. A GPIAS testing session consists of 3 blocks, lasting 36 minutes in total. The first block is the acclimation phase, lasting five minutes, where white noise is played from the “Cue” speaker for 5 minutes. Block two is the startle acclimation phase. To prevent outlier trials at the beginning of the testing session, 10 startle-only trials without any background sound are delivered to the animal. After block two, block three takes place, lasting 30 minutes. The 30 minute-long GPIAS session consisting of 300 individual trials (Five different background (BG) frequencies tested, for both GAP and NO_GAP conditions with 30 repeats for each BG frequency×GAP/NO_GAP condition pair). Each square represents one minute in the timeline. More specifically, five narrow-band noises (NBN)—9 kHz, 12.6 kHz, 16 kHz, 20.1 kHz, and 28.7 kHz are tested. All background bands are calibrated at 65 dB±3 dB Sound Pressure Level (SPL). The inter-trial interval is 6 seconds±3 s and the GAP or NO GAP startle occurs in the last three seconds of each trial.

FIG. 2B shows principle and schematic of gap pre-pulse inhibition of the acoustic startle reflex (GPIAS). Animal is startled when giving a loud sound (white noise, 20 ms, 116 dB). A silent gap in background 50 ms before startle stimulus will inhibit startle reflex in normal animal (Upper). R_G indicates GPIAS ratio, which is the ratio of the mean of the startle response in the GAP condition (b) to the mean of the startle response in the NO GAP condition (a). However, tinnitus animal shows defect in detecting the silent gap because of their tinnitus, thus, animal will show less inhibition of startle reflex (Lower), which means R_G will increase.

In particular, the trial is either GAP or NO GAP. During the GAP trial, the 50 ms gap in background NBN occurs 50-100 ms before the beginning of the 20 ms startle sound (116 dB). During NO GAP, such a gap in NBN is not present and NBN is continuous until startle sound. For each NBN (5 frequencies) and each GAP/NO GAP condition pair, there are 30 trials, thereby yielding 300 trials in total during block 3. Startle motion data from force sensors is transferred to TDT via band-pass filtering online (4-60 Hz, 48 dB/oct roll-off). The data are extracted in MATLAB script, and then further processed to obtain startle amplitudes for each trial, and outliers are removed. The GPIAS ratio of the mean startle response amplitude in gap trials/the mean startle response amplitude in no gap trials (R_G) is used as the criteria for the presence of tinnitus. A response of 1.0 means that the startle response amplitude is the same whether there is a gap or not, which means the mice cannot detect the gap in the background NBN. The farther the ratio is below 1.0 the better the startle inhibition.

Firstly, to verify that 1 hour of 12.6k Hz pure tone exposure is sufficient to induce chronic tinnitus in mice, the inventor compares the mean of GPIAS ratios on 3 consecutive days in two time periods—before the day of noise exposure (pre-NE), and 7 weeks after the day of noise exposure (7 weeks post-NE).

As shown in FIGS. 2C-2G, noise exposure induces chronic tinnitus in mice. In FIG. 2A-2E, R_G indicates GPIAS ratio, which is the ratio of the mean of the startle response in the GAP condition to the mean of the startle response in the NO GAP condition; pre-NE (noise exposure): before the day of noise exposure; 7 weeks post-NE: 7 weeks after the day of noise exposure. Bars indicate mean of GPIAS ratios on 3 consecutive days of 32 mice, plots represent mean of GPIAS ratio on 3 consecutive days of individual mice. Two-tailed Paired t test, *P≤0.05, **P≤0.01, ***P<0.001. ****P≤0.0001.

As can be seen from FIGS. 2C-2G, at the 7-week post-NE stage, where chronic tinnitus is expected to manifest, the mice exhibit a significant increase in GPIAS ratios at all frequencies. This finding suggests that the one-hour exposure to the 12.6k Hz pure tone led to the development of chronic tinnitus in mice across multiple frequencies. “Only mice that were noise-induced and showed relative startle ratios for gap detection above 0.7 were included in the tinnitus mice category” (Middleton, et al., Mice with behavioral evidence of tinnitus exhibit dorsal cochlear nucleus hyperactivity because of decreased GABAergic inhibition. Proc Natl Acad Sci USA. Vol. 108(18), pp. 7601-7606, 2011.). Since there is a systematic error, the example here uses a higher standard—Mouse whose GPIAS ratio 7 weeks after noise exposure is up to 0.8 is considered a tinnitus-positive mouse.

### Example 1

### Auditory Thalamocortical LTP can be Induced by High Frequency Stimulation (HFS) and CCK

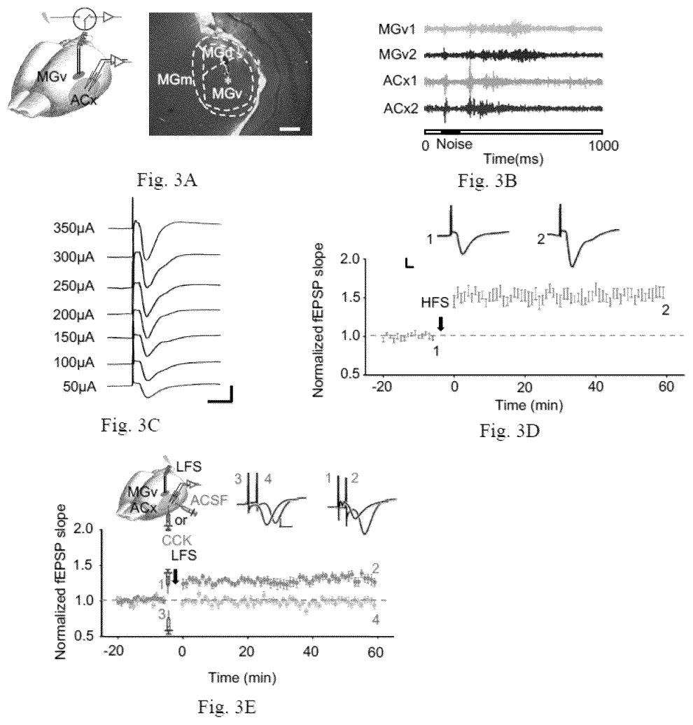

To establish the crucial role of cholecystokinin (CCK), expressed in the medial geniculate body (MGB), in thalamocortical long-term potentiation (LTP), a series of experiments is conducted. Initially, a high-frequency stimulation (HFS) is applied to the MGB using an in-vivo preparation to induce thalamocortical LTP. For the experimental setup, stimulation electrodes are positioned in medial geniculate body ventral division (MGv) and recording electrodes are positioned in the auditory cortex (ACx), as depicted in FIG. 3A. By utilizing these electrodes, neuronal responses to a noise-burst stimulus can be captured, as shown in FIG. 3B. In particular, FIG. 3B shows raw signal of the extracellular multiunit recording in MGv and ACx. 100 ms, 70 dB noise bursts are presented during the electrodes insertion. Robust sound responses are achieved when the electrodes reached the layer IV of the ACx and the MGv, respectively.

We quantify the field excitatory postsynaptic potential (fEPSP) elicited by electrical stimulation (ES) within the MGB (FIG. 3C). In particular, FIG. 3C shows the input-output relationship between stimulation intensity in MGv and evoked fEPSP in ACx. Negative current pulses, started from 50 μA and increased by 50 μA per step, are presented in MGv and the evoked fEPSPs are recorded from ACx to calculate the input-output relationship between the stimulation intensity and the level of the response. The baseline stimulation current is set at a value that could evoke 50% of the maximum response, while the HFS current is set at 75%.

The slope of fEPSPs is measured 1 h after the high-frequency stimulation (HFS) or low-frequency stimulation (LFS). This investigation reveals distinct responses to HFS and LFS of the MGv in relation to fEPSP. Specifically, HFS of MGv lead to a notable increase in fEPSP (FIG. 3D), indicative of a potentiated synaptic response.

FIG. 3E shows normalized fEPSP slopes before and after CCK (red) or ACSF (gray) injection in the ACx and LFS in the MGv (CCK: n=8, two-way ANOVA, p<0.001, post-hoc: Tukey test, p<0.001; ACSF: n=8, two-way ANOVA, post-hoc: Tukey test, p>0.05); and sample fEPSP waveforms before (1, 3) and after (2, 4) CCK and ACSF injection and LFS; scale bar: 5 ms, 0.1 mV. Conversely, LFS of MGv fails to elicit any significant change in fEPSP (FIG. 3E), suggesting a lack of synaptic potentiation under these conditions. However, it is observed that LFS, when combined with CCK application, lead to an enhancement in fEPSP (FIG. 3E). This suggests that the application of CCK, can indeed trigger neuroplastic changes within the thalamocortical pathway.

### Example 2

### HFS-Induced Thalamocortical LTP Enhances Neuronal Responses to a Natural Stimulus in the ACx

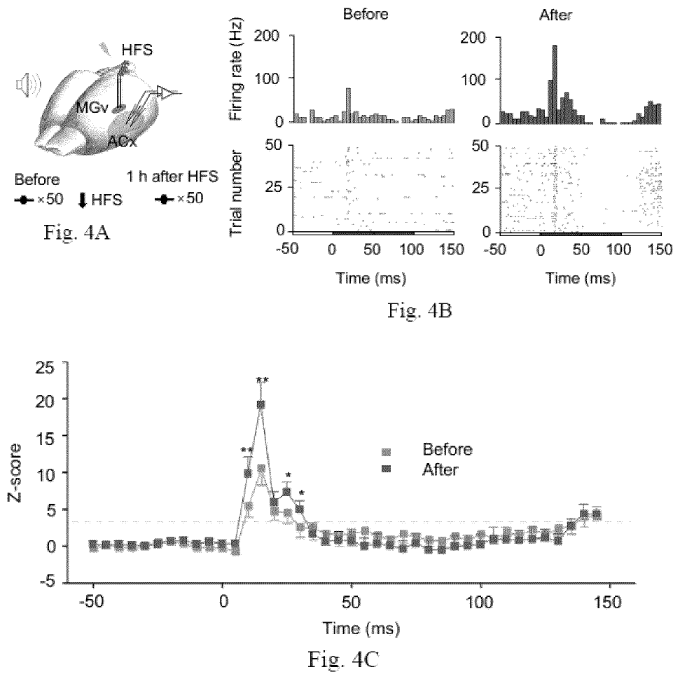

This example further determines whether the HFS-induced thalamocortical LTP also results in potentiated neuronal responses to a natural auditory stimulus within ACx, as depicted in FIG. 4A. LTP in auditory responses of 50 trials in the ACx is compared before and after the thalamocortical LTP induction. The example recording demonstrates that the firing rate of neuronal responses to the noise-burst stimulus in the ACx increased notably following the HFS, as illustrated in FIG. 4B. Z-score of neuronal responses to the noise bursts in the ACx significantly increase after HFS of MGv (FIG. 4C). This finding indicates that thalamocortical LTP can facilitate and strengthen the neuronal responses within the ACx specifically in response to the natural auditory stimulus.

### Example 3

Knockout of CCK Abolished HFS-Induced Thalamocortical LTP could be Rescued by CCK BR Agonist-HT-267

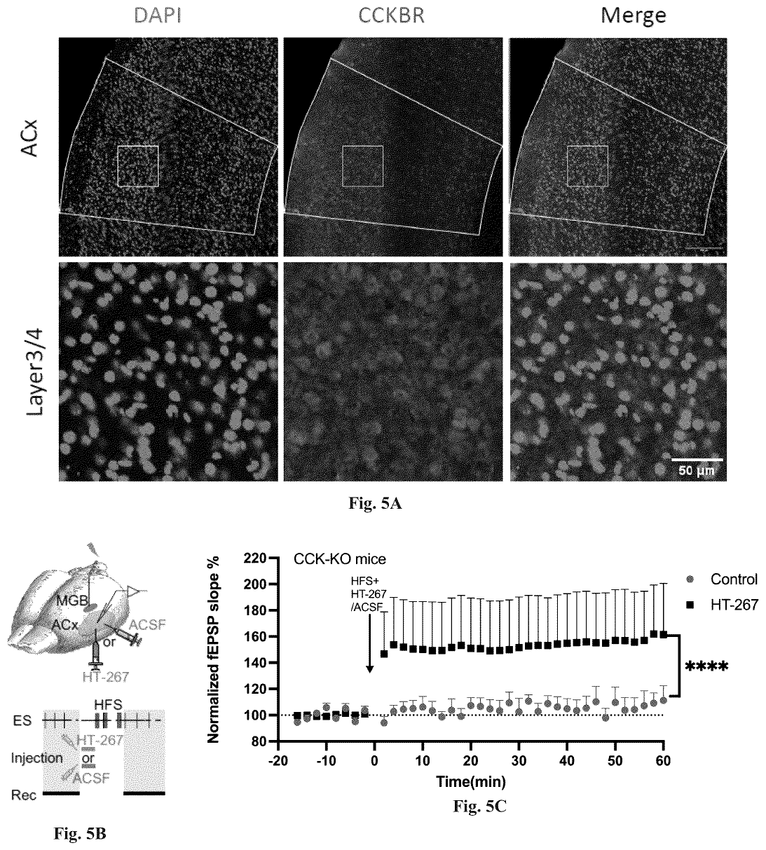

It is believed that the function of cholecystokinin (CCK) relies on its interaction with specific receptors, including the cholecystokinin type B receptor (CCKBR). CCKBR is abundantly expressed throughout the central nervous system (CNS). This receptor is believed to play a crucial role in mediating the effects of CCK and is involved in various physiological processes within the CNS. The inventors employ immunohistochemistry to investigate the expression patterns of CCKBR. Our findings reveal a rich distribution of CCKBR within ACx (FIG. 5A), which aligns with the in situ hybridization (ISH) data obtained from the Allen Mouse Brain Atlas.

We hypothesize that that the HFS-induced thalamocortical LTP is also dependent on CCK. Our results show that HFS-induced thalamocortical LTP is unable to be induced in cholecystokinin knockout (CCK-KO) mice, indicating an impairment in the ability to induce LTP in the absence of endogenous CCK. Nevertheless, the deficit in thalamocortical LTP can be rescued by administering HT-267, a specific agonist of CCKBR, via infusion into ACx (FIG. 5B).

As shown in FIG. 5C, HFS-induced thalamocortical LTP is abolished in CCK-KO mice, which means thalamocortical LTP is CCK-dependent. But this abolished LTP could be rescued by injection of HT-267 into ACx, which indicates HT-267 can induce thalamocortical neuroplasticity which is CCK-dependent. (HT-267: n=6; ACSF: n=8, two-way ANOVA, p<0.0001). The observed rescue effect of HT-267 highlights the significance of CCKBR activation in mediating the processes underlying thalamocortical plasticity. Its ability to restore the induction of LTP in the absence of endogenous CCK indicates that HT-267 can mimic the role of CCK and facilitate the necessary synaptic changes for LTP induction by specifically targeting CCKBR.

Moreover, this knowledge could potentially contribute to the development of therapeutic approaches targeting CCKBR signaling in neurological conditions characterized by dysfunction of thalamocortical plasticity like tinnitus.

### Example 4

### Therapy Using HT-267

In this example, our objective is to examine the potential benefits of a novel CCKBR agonist, HT-267, which possesses high affinity and stability, in tinnitus mice. We seek to determine whether this agonist could have a positive impact on tinnitus-related symptoms.

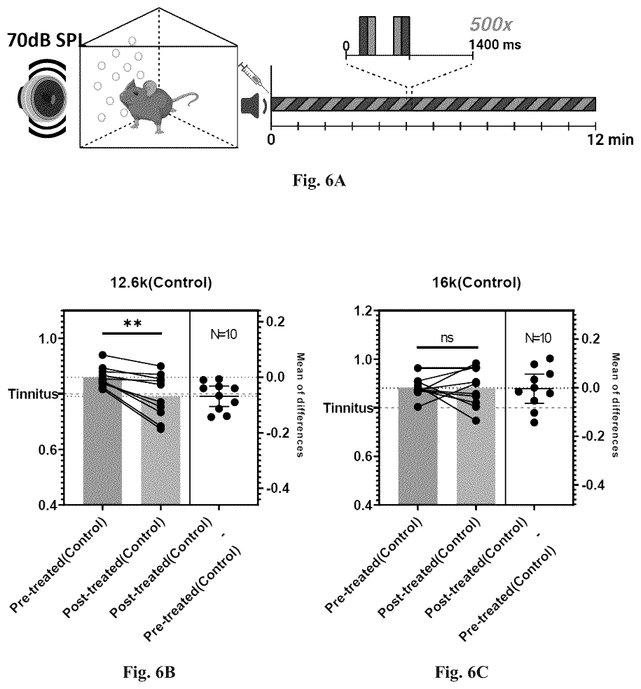

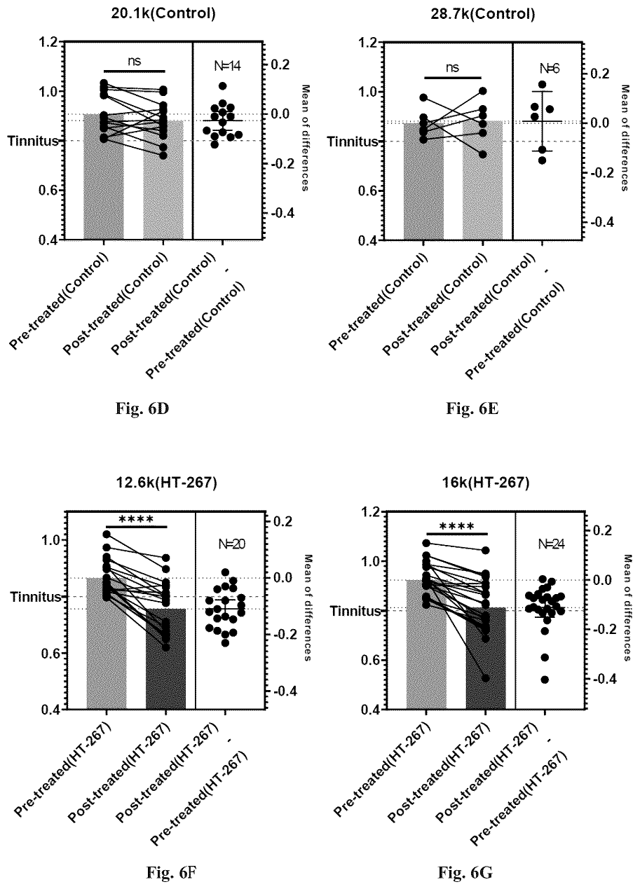

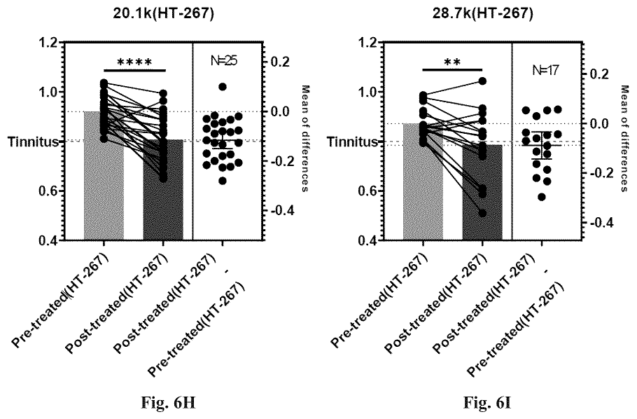

The mice that have developed chronic tinnitus are subjected to the following treatment (FIG. 6A). FIG. 6A shows schematic of HT-267 mediated sound therapy (Left), in which awake mouse injected either with HT-267 or vehicle is put into a plastic triangular prism. One lateral face with many small holes faces the speaker. The animal is subjected to 12-minute-long sound stimulation (70 dB SPL). The animal is injected either with HT-267 or vehicle as control (Right). 1400-ms zoom-in shows the repeating sound. In particular, the HT-267 treatment of tinnitus mice last for a week (4 days drug treated/3 days drug-free). On treatment day, after being injected intraperitoneally with HT-267 (0.2 mg/kg dosage) the mice are immediately subjected to a sound therapy which includes 500 sound trials. Each sound trial is 1400 ms which consists of 100 ms white noise (start at 150 ms and 600 ms) and 100 ms 5 merged frequencies (start at 250 ms and 500 ms). Mice are left in front of the speaker for around 12 minutes as soon as intraperitoneal injection until the sound is turned off. For the control group, saline is injected. The mean of the three-day GPIAS ratios before and after the treatment session is used for statistical analysis in tinnitus mice.

To evaluate the efficacy of HT-267, we compare the GPIAS ratios before and after HT-267-mediated sound therapy in mice with tinnitus. The changes of GPIAS ratio pre- and post-treated with control solution or HT-267 are shown in FIG. 6B to FIG. 6I. R_G indicates GPIAS ratio, which is the ratio of the mean of the startle response in the GAP condition to the mean of the startle response in the NO GAP condition. Bars indicate mean of GPIAS ratios on 3 consecutive days of mice in each group, plots represent the mean of GPIAS ratio on 3 consecutive days of individual mice. The left Y axis represents R_G (GPIAS ratio). Red dashed line represents R_G=0.8 (above 0.8 means tinnitus existence). Black dotted lines represent the mean of each group. The scatter on the third column of the graph represents the difference between the post-treated GPIAS ratio and the pre-treated GPIAS ratio for each mouse. Two-tailed Paired t test, *P≤0.05, **P≤0.01, ***P≤0.001. ****P<0.0001.

These results show that HT-267 combined with sound exposure significantly reduces the GPIAS ratio in mice with chronic tinnitus at multiple frequencies (12.6k, 16k, 20.1k, 28.7k) (FIGS. 6F-6I) in tinnitus mice. in contrast, the control solution combined with sound exposure has no significant effect on the GPIAS ratios of tinnitus mice at frequencies of 16k, 20.1k, and 28.7k Hz except at 12.6k Hz (FIGS. 6B-6E). These findings highlight the specific effectiveness of HT-267 in reducing the occurrence of tinnitus. Furthermore, the results indicate that sound therapy alone was able to relieve tinnitus in mice at the frequency of 12.6k Hz but not at higher frequencies, which suggests that sound therapy alone may have limitations in higher frequency tinnitus relief.

In summary, this example demonstrates that HT-267, as a novel CCKBR agonist, effectively reduces the occurrence of tinnitus when combined with sound exposure. Additionally, sound therapy alone is found to alleviate tinnitus in mice at a lower frequency. These results emphasize the potential of HT-267 as a promising therapeutic option for tinnitus management.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

All references specifically cited herein are hereby incorporated by reference in their entireties. However, the citation or incorporation of such a reference is not necessarily an admission as to its appropriateness, citability, and/or availability as prior art to/against the present invention.

What is claimed is:

1. A method of alleviation or therapy of tinnitus in a subject in need thereof, comprising the step of administrating a CCK-B receptor agonist to the subject.

2. The method of claim 1, wherein the CCK-B receptor agonist is selected from the group consisting of HT-267 comprising the following formula (I): 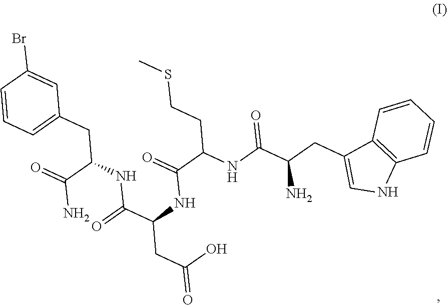 [SMILES: CSCCC(NC(=O)[C@H](N)Cc1c[nH]c2ccccc12)C(=O)N[C@@H](CC(=O)O)C(=O)N[C@@H](Cc1cccc(Br)c1)C(N)=O]
CCK-4 comprising the following formula (II): 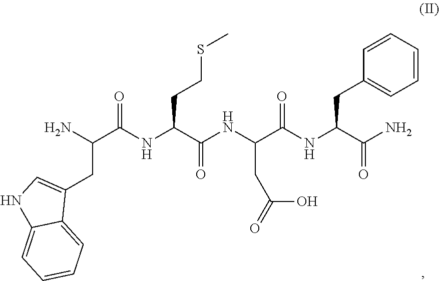 [SMILES: CSCC[C@H](NC(=O)C(N)Cc1c[nH]c2ccccc12)C(=O)NC(CC(=O)O)C(=O)N[C@@H](Cc1ccccc1)C(N)=O]
or a mixture thereof.

3. The method of claim 1, further comprising subjecting the subject to a sound exposure after administrating the CCK-B receptor agonist.

4. The method of claim 1, wherein the administrating step comprises a route selected from the group consisting of oral administration, intraperitoneal administration, intravenous administration, and a combination thereof.

5. The method of claim 1, wherein the CCK-B receptor agonist is administrated during a period of about a week.

6. The method of claim 1, wherein the CCK-B receptor agonist is administrated at a dosage of from about 0.1 mg/kg to about 5 mg/kg.

7. The method of claim 1, wherein the CCK-B receptor agonist is administrated for every two days.

8. The method of claim 1, wherein the CCK-B receptor agonist is in a dosage form selected from the group consisting of powder, solution, suspension, and a combination thereof.

9. The method of claim 1, wherein the subject is human.

10. The method of claim 1, wherein the CCK-B receptor agonist facilitates necessary synaptic changes for LTP induction by specifically targeting CCK-B receptor.

11. Use of a CCK-B receptor agonist for alleviation or therapy of tinnitus in a subject in need thereof.

12. The use of claim 11, wherein the CCK-B receptor agonist is selected from the group consisting of HT-267 comprising the following formula (I): 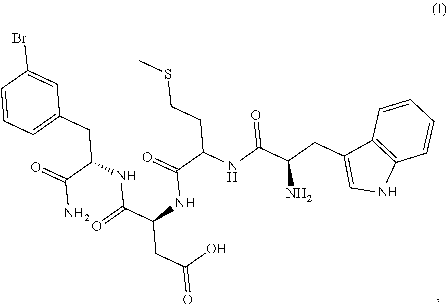 [SMILES: CSCCC(NC(=O)[C@H](N)Cc1c[nH]c2ccccc12)C(=O)N[C@@H](CC(=O)O)C(=O)N[C@@H](Cc1cccc(Br)c1)C(N)=O]
CCK-4 comprising the following formula (II): 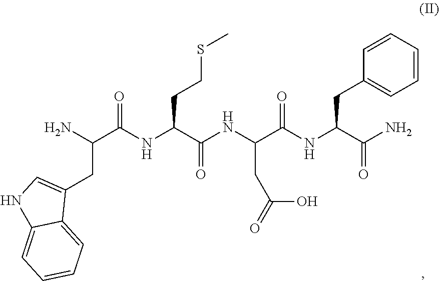 [SMILES: CSCC[C@H](NC(=O)C(N)Cc1c[nH]c2ccccc12)C(=O)NC(CC(=O)O)C(=O)N[C@@H](Cc1ccccc1)C(N)=O]
or a mixture thereof.

13. The use of claim 11, wherein the CCK-B receptor agonist is in a dosage form suitable for an administration route selected from the group consisting of oral administration, intraperitoneal administration, intravenous administration, and a combination thereof.

14. The use of claim 11, wherein the CCK-B receptor agonist is dosed in a concentration of about 0.02 wt. %.

15. The use of claim 11, wherein a sound exposure is applied in combination with the CCK-B receptor.

16. The use of claim 11, wherein the CCK-B receptor is administrated during a period of about a week.

17. The use of claim 11, wherein the CCK-B receptor is administrated at an effective amount of from about 0.1 mg/kg to about 5 mg/kg.

18. The use of claim 11, wherein the CCK-B receptor is administrated for every two days.

19. The use of claim 11, wherein the CCK-B receptor is in a dosage form selected from the group consisting of powder, solution, suspension, and a combination thereof.

20. The use of claim 11, wherein the subject is human.
